(19) United States
(12) Patent Application Publication
(10) Pub. No.: US 2004/0219547 A1
(43) Pub. Date: Nov. 4, 2004

(54) Biochip arrangement

(76) Inventors: Alexander Frey, Taufkirchen (DE); Franz Hofmann, Munchen (DE); Richard Johannes, Munchen (DE)

Correspondence Address: Jeffrey R Stone, Briggs & Morgan, 2200 IDS Center, Minneapolis, MN, 55402, US

(21) Appl. No.: 10/476,734

(22) PCT Filed: May 10, 2002
(86) PCT No.: PCT/DE02/01700

(30) Foreign Application Priority Data
May 10, 2001 (DE) 101-22-659.4

(51) Int. Cl. C12Q 1/68; C12M 1/34

(52) U.S. Cl. 435/6; 435/287.2

(57) ABSTRACT

A biochip arrangement, comprising a substrate, at least one sensor arranged on or in the substrate, and an electrically conductive permeation layer, arranged at a predetermined distance other than zero from the surface of the substrate and to which an electric voltage can be applied. As a result of the electrically conductive permeation layer being arranged at a distance other than zero from the surface of the substrate, there is no need for the permeation layer to be integrated on or in the chip. Therefore, production of the biochip arrangement eliminates the heretofore required method step of securing the permeation layer to the chip. This reducing cost and time required to produce the arrangement. The physical separation of the permeation layer from the substrate also brings with it the further advantage that the permeation layer does not take up any area on the substrate surface. Therefore, the invention avoids parasitic consumption of the surface area of the permeation layer to the detriment of the active sensor surface area. Thus, it is possible to provide the entire surface of the substrate with sensors, which bring with them an increased detection sensitivity. The active surface area on the substrate is increased in size compared to the prior art. This makes it possible to detect even lower concentrations of bio-molecules or to shorten the detection time.

The invention relates to a biochip arrangement.

In recent years, biotechnology and genetic engineering have become increasingly important. One fundamental technique of biotechnology and genetic engineering is to enable biological molecules, such as DNA (deoxyribonucleic acid) or RNA, proteins, polypeptides, etc., to be detected. In particular bio-molecules which code genetic information, in particular DNA molecules (deoxyribonucleic acid) are of great interest for numerous medical applications.

A DNA is a double helix composed of two linked helical individual chains, known as strands. Each of these strands has a sequence of bases, with the order of the bases (adenine, guanine, thymine, cytosine) defining the genetic information. DNA strands have the characteristic property of only binding very specifically to very specifically defined other molecules. Therefore, for a nucleic acid strand to dock onto another nucleic acid strand, it is imperative that the two molecules be complementary to one another. The two molecules clearly have to match one another in the same way as a key and the associated lock (known as the key-lock principle).

This principle, which is predetermined by nature, can be used for the selective detection of molecules in a liquid which is to be analyzed. The basic idea of a biochip sensor based on this principle consists in first of all what are known as capture molecules being applied (e.g. by means of micro-dispensing) to a substrate made from a suitable material and being immobilized thereon, i.e. permanently fixed to the surface of the biochip sensor. In this context, it is known for bio-molecules comprising thiol groups (SH groups) to be immobilized on gold surfaces.

A biochip sensor of this type having a substrate with capture molecules, which are sensitive, for example, to a specific DNA strand which is to be detected, bound to it is usually used to analyze a liquid for the presence of the DNA strand which is sensitive to the capture molecules. For this purpose, the liquid which is to be analyzed for the presence of a specific DNA strand is to be brought into active contact with the immobilized capture molecules. If a capture molecule and a DNA strand to be analyzed are complementary to one another, the DNA strand hybridizes to the capture molecule, i.e. is bound to it. If this binding causes the value of a physical variable which can be recorded by metrology to change in a characteristic way, the value of this variable can be measured, and in this way it is possible to detect whether or not a DNA strand is present in a liquid which is to be analyzed.

The principle described is not restricted to the detection of DNA strands. Rather, further combinations of capture molecules applied to the substrate and molecules which are to be recorded in a liquid to be analyzed are known. By way of example, nucleic acids can be used as capture molecules for peptides or proteins which have a nucleic acid-specific binding action. Furthermore, it is known to use peptides or proteins as capture molecules for other proteins or peptides which bind to the capture peptide or capture protein. The use of low-molecular chemical compounds as capture molecules for proteins or peptides which bind to these low-molecular compounds is also an important application. Low-molecular chemical compounds are chemical compounds of less than 1700 Dalton (molecular weight in grams per mole). Conversely, it is also possible to use proteins and peptides as capture molecules for any low-molecular compounds which may be present in a liquid which is to be analyzed.

Electronic detection methods are often used to detect the binding which has taken place between the capture molecule applied to the substrate and the molecule which is to be recorded and is present in the liquid to be analyzed. Detection methods of this type are becoming increasingly important in the industrial identification and assessment of new medicaments of organic or genetic engineering origin. These detection methods open up a wide range of applications, for example in medical diagnostics, in the pharmaceutical industry, in the chemical industry, in food analysis and in environmental and food technology.

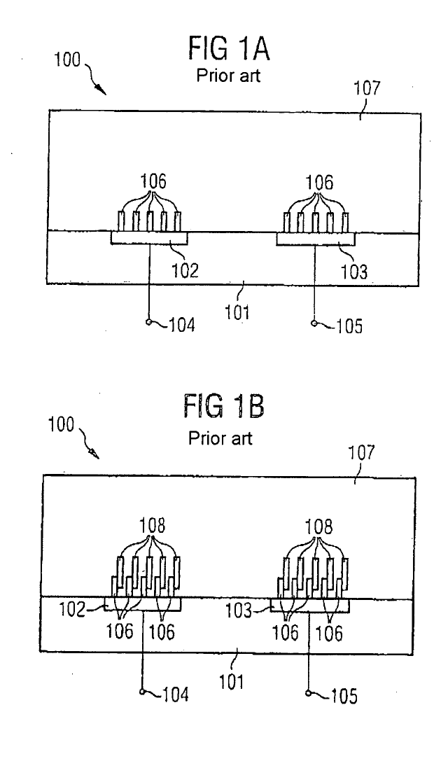

FIG. 1A and FIG. 1B show a biochip arrangement in accordance with the prior art which can be used for a DNA sensor in accordance with the principle described above. The biochip arrangement 100 includes a substrate 101, in the surface region of which a first electrode 102 and a second electrode 103 are arranged. The first electrode 102 is coupled to a first electrical contact 104. The second electrode 103 is coupled to a second electrical contact 105, it being possible for an electrical signal to be tapped off between the first electrical contact 104 and the second electrical contact 105. A multiplicity of capture molecules 106 are immobilized at the surface of the first electrode 102 and at the surface of the second electrode 103. The first electrode 102 and the second electrode 103 are often made from a gold material, and the capture molecules 106 are often immobilized on the first and second electrodes 102, 103 in the form of a gold-sulfur coupling. Many bio-molecules have sulfur atoms in their end sections, for example what are known as thiol groups (SH groups): the gold-sulfur material pairing has particularly favorable coupling properties. Furthermore, FIG. 1A shows an electrolytic liquid 107 which is to be analyzed and may contain DNA strands 108 which are complementary to the capture molecules 106.

If the capture molecules 106 undergo a specific binding reaction with a molecule present in the liquid 107 to be analyzed in accordance with the key-lock principle (according to which only those molecules in the liquid 107 to be analyzed for which the capture molecules have a sufficient binding specificity can be bound by the capture molecules 106), the molecule (e.g. a DNA strand 108) in the liquid 107 to be analyzed is specifically bound by the capture molecules 106. If not, the molecule in the liquid 107 to be analyzed is not bound by one of the capture molecules 106. If DNA strands 108 with a base sequence which is complementary to the base sequence of the capture molecules 106 (i.e. of the DNA probe molecules) are present in the electrolytic liquid 107 to be analyzed, these DNA strands 108 hybridize with the DNA probe molecules 106. This is shown in FIG. 1B.

Hybridization of a DNA probe molecule 106 with a DNA strand 108 occurs only if the base sequences of the respective DNA probe molecule 106 and of the matching DNA strand 108 are complementary to one another. If not, no hybridization takes place. Therefore, a DNA probe molecule 106 with a predetermined base sequence is in each case only able to bind, i.e. hybridize with, very specific DNA strands, namely DNA strands with a complementary base sequence. The term hybridization denotes the binding of DNA strands to capture molecules.

Successful hybridization of DNA strands 108 to capture molecules 106 has a characteristic effect on an electrical signal which can be tapped off between the first electrical contact 104 and the second electrical contact 105. The DNA strands 108 and the capture molecules 106 are as far as possible electrically nonconductive and clearly electrically shield the first electrode 102 and the second electrode 103, respectively. As a result, the capacitance between the first electrode 102 and the second electrode 103 changes. The change in capacitance is used as a measurement variable for recording DNA molecules. This is because if the liquid to be analyzed contains molecules which are to be detected, and if these molecules have hybridized with the capture molecules on the surface of the electrodes, the value, which can be recorded by metrology, of the capacitance of the electrodes 102, 103, which can be interpreted as capacitor surfaces, changes.

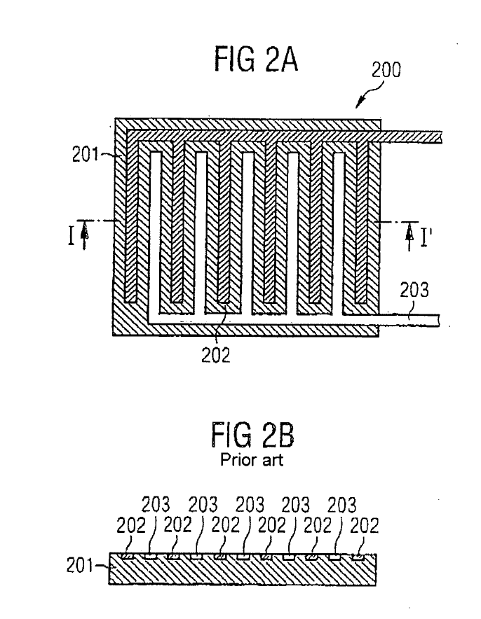

FIG. 2A shows a plan view of a biochip arrangement 200 with interdigitated electrodes 202, 203. Furthermore, FIG. 2B shows a cross section through the biochip arrangement 200 shown in FIG. 2A on line I-I′. The biochip arrangement 200 includes a substrate 201, a first interdigitated electrode 202 and a second interdigitated electrode 203. The first and second interdigitated electrodes 202, 203 shown in FIG. 2A, FIG. 2B form an approximately meandering surface structure on the substrate.

However, the biochip arrangements described in accordance with the prior art have a number of drawbacks. Biological molecules, such as for example DNA strands or proteins, are often only present in very low concentrations (millimolar, or sometimes even only micromolar). Therefore, the response time of the DNA sensors shown in FIG. 1A, FIG. 1B, FIG. 2A, FIG. 2B is very high.

The term response time is understood as meaning a characteristic time which it is necessary to wait before molecules which are to be detected have been bound to capture molecules in sufficient number and consequently a change in the capacitance which can be detected by metrology has occurred.

Since the hybridization, which is a precondition for the biosensor to function, only occurs after a considerable response time, the biochip arrangement of the prior art is of only limited use under practical laboratory conditions. Rapid detection of molecules is regularly desired. In many cases, bio-molecules which are to be detected, for example unstable mutants of proteins, are denatured with time constants of just a few hours and less. Therefore, the slow response time of the DNA sensor described which is known from the prior art is extremely disadvantageous and restricts the potential applications of the device.

Furthermore, the sensitivity of the biochip arrangement according to the prior art is not sufficiently high, a fact which is likewise associated with the low concentration of the bio-molecules to be detected in the vicinity of the electrodes provided with capture molecules.

[1] discloses a biochip arrangement which makes it possible for a sufficiently large number of DNA molecules to be docked to the capture molecules within a sufficiently short time even with low DNA concentrations. According to [1], this is achieved by what is known as a permeation level being applied direct to the chip. The permeation level which is known from [1] has an electrically conductive layer which is surrounded by a porous protective layer. An electric voltage can be applied to the electrically conductive layer.

The biochip arrangement described in [1] makes use of the fact that many bio-molecules, such as proteins or DNA, are electrically charged. For example, in the case of proteins, certain amino acids on the protein surface are positively charged, and others negatively charged, as a function of the pH of the surrounding medium, so that overall proteins can be either positively or negatively electrically charged. Also, DNA molecules regularly have a negative electrical charge at physiological pH values (pH 6 to pH 9).

If an electric voltage with a suitable sign is applied to the permeation layer, the bio-molecules move toward the permeation layer as a function of their electrical charge on account of electrophoresis, in order to accumulate in the immediate vicinity of the permeation layer. The principle of electrophoresis in connection with bio-molecules is described, for example, in [2]. As has already been mentioned above, DNA molecules are generally negatively charged. If a positive voltage is applied to the permeation layer, an electrically attracting force is applied to the DNA molecules and the DNA molecules will accumulate in the vicinity of the permeation layer. Consequently, the concentration of the DNA strands increases in the vicinity of the permeation level and therefore in an area surrounding the active sensor surface. As a result of diffusion, the DNA strands pass to the capture molecules. The increased DNA concentration means that the hybridization as a result of the electric voltage applied to the permeation level then takes place more quickly and more effectively.

However, it should be emphasized that DNA molecules may be broken down if they come into direct contact with free charge carriers at the surface of an electrode. Therefore, DNA molecules and other sensitive bio-molecules may be destroyed if they come into contact with the electrically conductive layer of the permeation layer. According to the biochip arrangement which is known from [1], a porous protective layer is provided around the electrically conductive core layer of the permeation layer. This porous protective layer around the electrically conductive core of the permeation layer is only pervious to ions of the electrolyte, whereas molecules above a predetermined size cannot penetrate through the porous protective layer. Therefore, biological macromolecules, such as DNA strands or proteins, cannot penetrate through the porous protective layer, so that the sensitive bio-molecules are protected from direct contact with the electrically conductive layer of the permeation layer by the porous protective layer. This protects the bio-molecules from being destroyed.

The biochip arrangement which is known from [1] is also subject to a number of drawbacks. For example, integration of the permeation level directly on the chip is technologically difficult and expensive. To ensure correct functioning, a sufficiently large area of the chip has to be provided with the permeation layer. This surface area is occupied at the expense of the interdigitated electrodes. Therefore, the provision of the permeation layer on the chip reduces the active sensor area which is available for the interdigitated electrodes.

Therefore, the active surface area at which capture molecules can be immobilized is reduced by the presence of the permeation layer. This entails a loss of detection sensitivity. The response time which it is necessary to wait for the molecules which are to be detected to hybridize with the capture molecules is thereby increased.

Furthermore, [3] describes a method for carrying out reactions between at least two reaction partners, in particular bio-molecules, in which at least one bio-molecule passes through reaction regions of different reaction conditions, and at least one reaction partner, for example a feature of a biochip, is immobilized, and in which the reaction mixtures are moved hydrodynamically.

The invention is based on the problem of providing a biochip arrangement with an increased detection sensitivity.

The problem is solved by a biochip arrangement having the features described in the independent patent claim.

The biochip arrangement of the invention includes a substrate, at least one sensor arranged on or in the substrate, and an electrically conductive permeation layer, which is arranged at a predetermined distance other than zero from the surface of the substrate and to which an electric voltage can be applied.

As a result of the electrically conductive permeation layer being arranged at a distance other than zero from the surface of the substrate, according to the invention there is no need for the permeation layer to be integrated on or in the chip. Therefore, production of the biochip arrangement according to the invention is simplified in terms of process engineering compared to the prior art. When producing the biochip arrangement, compared to the prior art in particular the method step of securing the permeation layer to the chip is eliminated. This reduces the costs and time required to produce the arrangement.

The physical separation of the permeation layer from the substrate also brings with it the further advantage that the permeation layer does not take up any area on the substrate surface. Therefore, the invention avoids parasitic consumption of the surface area of the permeation layer to the detriment of the active sensor surface area. According to the biochip arrangement of the invention, it is possible to provide the entire surface of the substrate with sensors, which bring with them an increased detection sensitivity. The active surface area on the substrate is increased in size compared to the prior art. This makes it possible, according to the invention, to detect even lower concentrations of bio-molecules or to shorten the detection time.

According to a preferred exemplary embodiment of the invention, the biochip arrangement also includes a spacer which is arranged between the substrate and the permeation layer and the thickness of which is equal to the predetermined distance between the permeation layer and the surface of the substrate.

Therefore, the thickness of the spacer can be used to accurately predetermine the distance between the permeation layer and the surface of the substrate, the spacer preferably having a thickness of between approximately 1 μm and approximately 2 μm. The thickness of the spacer can be adjusted flexibly to meet the requirements of the individual case. The spacer can be made from any desired, low-cost material, which keeps the production costs of the biochip arrangement low.

Furthermore, the biochip arrangement may include a delimiting device, in which case the delimiting device is arranged along a continuous path on the permeation layer, in such a manner that the delimiting device and the permeation layer form a cavity.

A liquid which is to be analyzed can easily be introduced into the cavity formed by the delimiting device and the permeation layer. The dimensions of the delimiting device can be flexibly adjusted to the volumes of a liquid to be analyzed which are available in the individual situation. Therefore, the biochip arrangement of the invention is suitable even for applications with very small volumes, as often occur in biochemistry.

The delimiting device may also be made from any desired material, for example from an inexpensive plastic or Plexiglass material. This keeps the costs of producing the biochip arrangement low.

According to preferred configurations of the invention, both the spacer and the delimiting device, independently of one another, are designed substantially in the form of hollow cylinders. It is preferable for the spacer and/or the delimiting device to be made from an electrically nonconductive material. This material may in particular be one or a combination of the materials glass, Plexiglass, polyimide, polycarbonate, polyethylene, polypropylene or polystyrene.

Furthermore, the biochip arrangement of the invention may include at least one further spacer, in which case each of the further spacers is arranged between the substrate and the permeation layer, with the thickness of each of the further spacers being equal to the predetermined distance between the permeation layer and the surface of the substrate. Obviously, the further spacers can act as supporting devices for the permeation layer. According to a preferred embodiment of the invention, the permeation layer is arranged on the spacer which is substantially in the form of a hollow cylinder or ring. If at least one further spacer is provided in the interior of the annular spacer, the thickness of this further spacer being equal to the predetermined distance between the permeation layer and the surface of the substrate, the at least one further spacer can provide additional mechanical stability to the permeation layer. In this way, the biochip arrangement can be additionally stabilized, so that it is suitable for robust laboratory use.

According to one configuration of the invention, the at least one sensor has at least one electrode, it being possible for each of the electrodes to be coupled to electrical contact-making means. Furthermore, the biochip arrangement according to a configuration of the invention includes a multiplicity of capture molecules, which are coupled to at least one of the electrodes. In addition, the biochip arrangement may include at least one electrical contact-making means, in which case at least one of the electrodes is coupled to at least one of the contact-making means, so that at least one signal can be tapped off at the electrical contact-making means.

The capture molecules are clearly immobilized on the surface of the electrodes. If a liquid which is to be analyzed and may contain molecules which are complementary to the capture molecules immobilized at the electrode is introduced into the biochip arrangement, these complementary molecules, for example DNA strands, hybridize with the capture molecules. This has a characteristic influence on a parameter which can be recorded electrically, for example the capacitance which can be taken off between the electrodes. In this way, the biochip arrangement of the invention can be used as a sensor for bio-molecules, for example as a DNA sensor.

Each of the at least one electrode is made from an electrically conductive material, for example a metallic material. The electrode is preferably made from gold material. The capture molecules may be nucleic acids (DNA or RNA strands), peptides, proteins, low-molecular compounds or alternatively any other suitable molecule.

The capture molecules are preferably immobilized on the at least one electrode by means of a gold-sulfur coupling. For this purpose, it is necessary for the capture molecules to include a sulfur-containing group, for example a thiol group (SH), in one of their end sections. However, the biochip arrangement is in no way restricted to the gold-sulfur material pairing. It is also possible for any other suitable material pairing to be used to immobilize the capture molecules on the electrode. A precondition is for a chemical bond to form between the molecules and the conductor material. In addition to the abovementioned gold-thiol bond, there are also numerous further suitable combinations, for example thiol groups also bond to platinum or silver, trichlorosilanes adhere to various oxides, which may be provided as thin surface films on the electrically conductive layer. However, trichlorosilanes (SiCl₃ groups) also adhere to silicon, aluminum and titanium.

Furthermore, the biochip arrangement may include a reference electrode, in such a manner that an electric voltage can be applied between the permeation layer and the reference electrode. The reference electrode is immersed in the liquid which is to be analyzed (generally an electrolytic liquid).

To enable an electric voltage to be applied between the permeation layer and the reference electrode, the permeation layer has a core which is made from an electrically conductive material. The electrically conductive material of the permeation layer may in particular be a metal or a semiconductor. The electrically conductive material of the permeation layer is preferably gold.

As has been indicated above, bio-molecules are often very sensitive macromolecules which are only sufficiently stable under certain biological-chemical or physical conditions. For example, proteins are denatured above a certain temperature or outside a certain range of pH values. DNA strands are particularly sensitive to free electrical charges. Therefore, DNA strands may be broken down if they come into direct contact with a metallic electrode if free electrical charges are present at this metallic electrode. For this reason, the core of the permeation layer made from an electrically conductive material is surrounded by a covering made from a porous material.

The porous material of the permeation layer has pores of a predeterminable size, such that molecules whose size is less than or equal to the predetermined pore size can diffuse through the porous material, whereas molecules whose size exceeds the predetermined pore size cannot diffuse through the porous material. This enables the electrolyte molecules, which are usually of small volume, to diffuse through the porous protective layer to the electrically conductive layer, whereas bio-molecules which are sensitive to free electrical charge carriers, such as DNA strands, cannot diffuse through the porous protective layer on account of the large size of their molecules. Consequently, the sensitive bio-molecules are decoupled from the electrically conductive layer of the permeation layer and protected from free electrical charges.

To record biological macromolecules, first of all the liquid to be analyzed is to be introduced into the cavity defined by the delimiting device and the permeation layer. If an electric voltage of suitable sign is then applied between the permeation layer and the reference electrode, an electric field is generated in the liquid which is to be analyzed. This electric field applies an electrical force to the bio-molecules contained in the liquid to be analyzed, if these molecules are electrically charged. For example, if the permeation layer is at an electrically positive potential, the DNA strands, which are usually negatively charged, are electrically attracted by the permeation layer. As a result, the concentration of the DNA strands compared to the mean concentration of the DNA strands in the liquid introduced increases in the vicinity of the permeation layer.

The at least one sensor on the surface of the substrate is arranged at a distance from the permeation layer which, although different from zero, can be set to be sufficiently small. The increase in concentration of the molecules to be detected reaches a maximum close to the permeation layer and drops at increasing distance from the permeation layer. The smaller the distance is set to be, the greater the effect of the increase in concentration brought about by the permeation layer on the concentration of the bio-molecules at the active sensor surface becomes. Therefore, according to the invention the concentration of the bio-molecules to be detected is also increased in the immediate vicinity of the sensors. This increase in concentration of the bio-molecules to be detected is associated with an increase in the detection sensitivity and/or with a reduction in the characteristic response time required for hybridization.

The biochip arrangement of the invention is of very simple structure, so that it can be produced at low cost and within little time.

The permeation layer of the biochip arrangement is oriented in such a manner that the bio-molecules can penetrate through it. According to the invention, this is necessary in order for the molecules which are to be detected to be brought into direct active contact with the capture molecules arranged at the surface of the sensors. This can be achieved by the permeation layer of the invention being formed as a grid. Clearly, a grid of this type comprises wires running in two directions which are orthogonal to one another. The meshes of the grid which are defined by these wires are to be selected to be sufficiently large to enable the bio-molecules which are to be detected to be brought into direct contact with the capture molecules arranged on the electrodes. In other words, the bio-molecules have to be able to pass through the meshes of the grid. The wires of the grid are preferably arranged at a distance of approximately 100 nm from one another.

According to a preferred exemplary embodiment, the wires may be made from an electrically conductive core and a porous covering layer arranged around it. By way of example, first of all a grid can be made from a metallic material, and this grid can then be immersed in a bath which contains the material which is able to form a porous covering layer around the grid. A grid of this type can be produced at low cost and without difficulty.

According to one configuration of the invention, the biochip arrangement may include a plurality of electrically conductive permeation layers, which are arranged at predetermined distances from one another and substantially parallel to one another, in which case an electric voltage can be applied to each of the permeation layers. If a plurality of permeation layers connected in series are used instead of a single permeation layer and suitable electric voltages are connected to them, the concentration of the molecules to be detected, for example DNA strands, can be gradually increased from permeation level to permeation level. This additionally increases the sensitivity of the DNA sensor and reduces the response time required for hybridization.

The biochip arrangement can be used, for example, as a DNA sensor. A DNA sensor of this type includes, for example, a substrate, on which substrate a polyimide ring is provided as a spacer with respect to the active contact level. The contact level is the surface of the substrate which is provided with at least one sensor. As has already been explained above, a sensor of this type may be configured as a gold electrode with capture molecules immobilized thereon. The thickness of the polyimide ring is, for example, 1 μm to 2 μm. A maximum stability of the permeation grid which is formed on the polyimide ring can be achieved if additional spacers are provided as supporting points made from polyimide. According to the exemplary embodiment described, the delimiting device used is a Plexiglass tube. The Plexiglass tube may be adhesively bonded to the permeation grid, and this Plexiglass tube which has been adhesively bonded to the permeation grid can be pressed onto the polyimide ring and adhesively bonded to it. The polyimide ring is secured to the surface of the substrate, for example by adhesive bonding. The permeation grid is arranged very close to the contact level of the capture molecules. If an electric voltage of suitable sign is then applied between the permeation grid and a reference electrode, electrophoresis causes the concentration of DNA strands contained in the liquid to be analyzed to increase in the vicinity of the permeation level. A gradual increase in concentration from permeation layer to permeation layer can be achieved as a result of a plurality of permeation levels arranged parallel to one another being connected in series and suitable voltages being applied to each of the permeation levels.

Exemplary embodiments of the invention are illustrated in the figures and are explained in more detail in the text which follows. In the figures:

FIG. 1A shows a cross-sectional view of a biochip arrangement in accordance with the prior art,

FIG. 1B shows another cross-sectional view of a biochip arrangement in accordance with the prior art,

FIG. 2A shows a plan view of a biochip arrangement with interdigitated electrodes in accordance with the prior art,

FIG. 2B shows a cross-sectional view on line I-I′ of the biochip arrangement with interdigitated electrodes in accordance with the prior art shown in FIG. 2A,

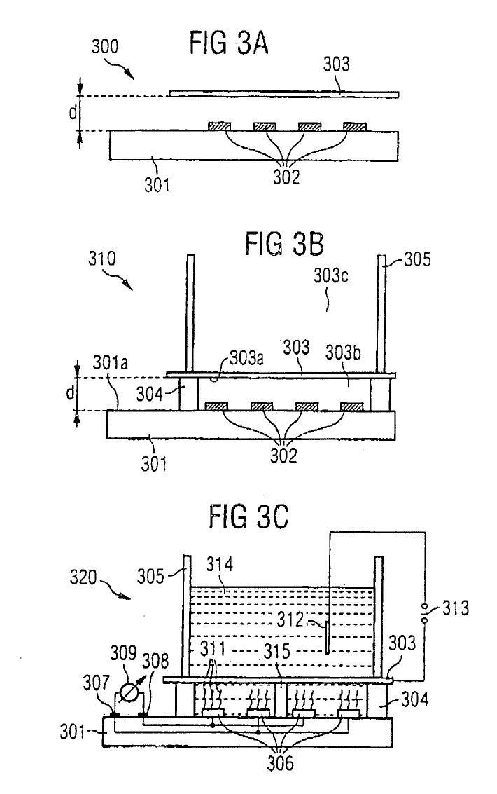

FIG. 3A shows a cross-sectional view of a biochip arrangement in accordance with a first exemplary embodiment of the invention,

FIG. 3B shows a cross-sectional view of a biochip arrangement in accordance with a second exemplary embodiment of the invention,

FIG. 3C shows a cross-sectional view of a biochip arrangement in accordance with a third exemplary embodiment of the invention,

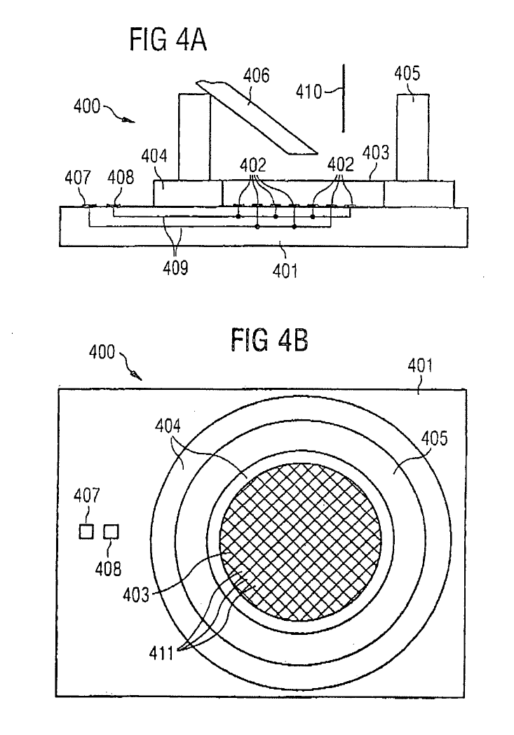

FIG. 4A shows a cross-sectional view of a biochip arrangement in accordance with a fourth exemplary embodiment of the invention,

FIG. 4B shows a plan view of the biochip arrangement shown in FIG. 4A in accordance with the fourth exemplary embodiment of the invention.

FIG. 3A shows a biochip arrangement in accordance with a first exemplary embodiment of the invention. The biochip arrangement 300 includes a substrate 301, at least one sensor 302 arranged on or in the substrate 301 and an electrically conductive permeation layer 303, which is arranged at a predetermined distance other than zero (this distance is indicated by a double arrow denoted by “d” in FIG. 3A) from the surface of the substrate 301 and to which an electric voltage can be applied.

According to the exemplary embodiment shown in FIG. 3A, the biochip arrangement 300 has four sensors 302. The four sensors 302 are applied to the substrate 301. Alternatively, according to the invention it is possible for the sensors 302 to be arranged in a surface section of the substrate 301, for example the sensors 302 may be introduced as integrated circuit elements in a semiconductor substrate 301. The substrate 301 may, for example, be a semiconductor wafer (for example made from silicon), may be a silicon dioxide plate or a plate made from any other suitable material. For example, the substrate 301 may also, for example, be a small die of glass.

In accordance with the exemplary embodiment of the biochip arrangement 310 shown in FIG. 3B, the biochip arrangement, in addition to the biochip arrangement 300 shown in FIG. 3A, has a spacer 304 which is arranged between the substrate 301 and the permeation layer 303 and the thickness d (cf. FIG. 3B) of which is equal to the predetermined distance between the permeation layer 303 and the surface of the substrate 301. By suitably selecting the thickness of the spacer 304, it is possible to predetermine the distance between the permeation layer 303 and the active sensor surface on the substrate 301. According to this exemplary embodiment, the spacer 304 has a thickness, d, calculated starting from the surface 301a of the substrate 301 to the lower surface 303a of the permeation layer 303, of approximately 1 μm to 2 μm.

The spacer 304 is preferably arranged along a continuous closed path on the substrate 301, in such a manner that a cavity 303b is formed by the spacer 304 and the substrate 301. In particular, the spacer 304 may be substantially in the form of a hollow cylinder. Alternatively, the spacer 304, when seen in plan view, may also be configured as a rectangle or other geometric figure. Alternatively, the shape of the spacer 304 may be whatever shape is most favorable for a specific application. The spacer 304 is preferably made from an electrically nonconductive material, for example from one or a combination of the materials glass, Plexiglass, polyimide, polycarbonate, polyethylene, polypropylene or polystyrene. The spacer 304 is secured to the substrate 301. The spacer 304 may in particular be adhesively bonded to the substrate 301. Alternatively, it is possible for the spacer 304 to be applied to the substrate surface by means of a semiconductor technology process. For example, it is possible for a layer of the material from which the spacer 304 is to be produced to be applied to the substrate surface and for the spacer 304 to be formed on the substrate 301 by means of photographic patterning.

Furthermore, the spacer 304 is secured to the permeation layer 303. According to this exemplary embodiment, the spacer 304 is adhesively bonded to the permeation layer 303. The biochip arrangement 310 which is shown in FIG. 3B also includes a delimiting device 305, the delimiting device 305 being arranged along a continuous path on the permeation layer 303, in such a manner that a further cavity 303c is formed by the delimiting device 305 and the permeation layer 303.

According to this exemplary embodiment, the delimiting device 305 is configured substantially in the form of a hollow cylinder or ring. The delimiting device 305 is made from an electrically nonconductive material, such as for example glass, Plexiglass, polyimide, polycarbonate, polyethylene, polypropylene or polystyrene. Alternatively, the delimiting device 305 may be made from a combination of the abovementioned materials, or may alternatively be made from any other suitable material. Since a cavity is formed by the permeation layer 303 and the delimiting device 305, a liquid which is to be analyzed can be introduced into this cavity. By way of example, the liquid which is to be analyzed can be introduced into the cavity using a cannula or a pipette. As shown in FIG. 3B, the delimiting device 305 also has a characteristic height. By suitably selecting this height of the delimiting device 305, the biochip arrangement 310 can be flexibly adapted to the requirements of the specific case. Often, biological specimens which are to be analyzed using a biochip arrangement 310 are only present in small volumes. The delimiting device 305 can be selected to have a suitable height in accordance with a predetermined volume of a liquid which is to be analyzed. The delimiting device 305 is secured to the permeation layer 303 by virtue of being adhesively bonded to the permeation layer 303.

FIG. 3C shows a biochip arrangement 320 in accordance with a further exemplary embodiment of the invention.

In accordance with the biochip arrangement 320 shown in FIG. 3C, the at least one sensor 302 from FIG. 3A, FIG. 3B has at least one electrode 306, it being possible for each electrode 306 to be coupled to electrical contact-making means 307, 308. In accordance with the exemplary embodiment shown in FIG. 3C, each of the electrodes 306 is coupled to a first electrical contact-making means 307 or to a second electrical contact-making means 308. As shown in FIG. 3C, a signal can be tapped off between the first electrical contact-making means 307 and the second electrical contact-making means 308. In accordance with the exemplary embodiment shown in FIG. 3C, this signal is recorded by a means for recording an electrical signal 309. The means for recording an electrical signal 309 may be a voltmeter. Furthermore, the biochip arrangement 320 includes a multiplicity of capture molecules 311, which are coupled to the electrodes 306. Furthermore, the biochip arrangement shown in FIG. 3C includes a reference electrode 312. An electric voltage 313 can be applied between the reference electrode 312 and the permeation layer 303. The magnitude and sign of the electric voltage 313 can be selected as desired. FIG. 3C diagrammatically depicts an electrolytic liquid 314 which is to be analyzed and which can be analyzed for the presence of a specific bio-molecule, for example a specific DNA strand, by means of the biochip arrangement 320.

Bio-molecules which are to be detected may be present (although are not shown in FIG. 3C) in the liquid 314 to be analyzed. Furthermore a liquid 314 which is to be analyzed can be introduced into the biochip arrangement 320 by means of an introduction device (not shown in FIG. 3C). An introduction device of this nature may, for example, be a pipette or a cannula.

Furthermore, FIG. 3C shows a further spacer 315. This further spacer, like the spacer 304, is arranged between the substrate 301 and the permeation layer 303. The thickness of the further spacer 315 is equal to the predetermined distance between the permeation layer 303 and the surface of the substrate 301, i.e., as indicated in FIG. 3C, the spacer 304 predominantly performs two functions. Firstly, the spacer 304 maintains the distance between the substrate 301 and the permeation layer 303 which can be predetermined by its thickness. Furthermore, the spacer 304 is used to mechanically stabilize the permeation layer 303 along the circumference of the, for example, substantially circular permeation layer 303. If the spacer 304 is substantially in the form of a ring or hollow cylinder, it may not provide sufficient mechanical stability to the permeation layer 303 in a central section. In view of this, according to the exemplary embodiment shown in FIG. 3C, the further spacer 315 is provided, stabilizing the substantially circular permeation layer 303 in a central section. As an alternative to the exemplary embodiment shown in FIG. 3C, it is also possible to provide a plurality of further spacers 315 in order to mechanically stabilize the permeation layer 303 at further locations. The at least one further spacer 315 can also be used to maintain the constant distance between the substrate 301 and the permeation layer 303 in a central section of the permeation layer 303. This may be necessary if the permeation layer 303 sags down under the force of gravity on account of its own weight or the weight of the liquid 314 to be analyzed which is arranged on the permeation layer 303. In this case, the at least one further spacer 315 can maintain the substantially planar structure of the permeation layer 303.

The permeation layer 303 shown in FIG. 3C has a core which is made from an electrically conductive material. Furthermore, the permeation layer 303 has a covering which surrounds the core and is made from a porous material. The porous material of the permeation layer 303 has pores of a predetermined size, such that molecules whose size is less than or equal to the predetermined pore size can diffuse through the porous material whereas molecules whose size exceeds the predetermined pore size cannot diffuse through the porous material.

To protect the sensitive bio-molecules, for example DNA strands, which may be present in a liquid 314 which is to be analyzed from being destroyed, the core, which is made from an electrically conductive material, of the permeation layer 303 is surrounded by the porous protective layer. Small molecules and ions can penetrate through this protective layer, whereas large molecules, such as the sensitive bio-molecules which are to be analyzed, cannot penetrate through the protective layer. Since the pore size can be set by selecting the material used for the porous covering of the permeation layer 303, it is possible to determine which molecules are able to diffuse through the porous protective layer and which are not.

The electrically conductive material of the permeation layer 303 is preferably a metal or a semiconductor. However, it may also be any other conductive material, for example electrically conductive polymers. The electrically conductive material of the permeation layer 303 is preferably gold material.

In the biochip arrangement 320, the permeation layer 303, according to this exemplary embodiment of the invention, is configured in the form of a grid. A grid of this type is formed from electrically conductive wires held in parallel in two mutually orthogonal directions, these wires being surrounded by a porous material. The wires of the grid, which, for example, are arranged at intervals of approximately 100 nm, form meshes, the dimensions of which are preset in such a way that the bio-molecules to be detected which may be present in the liquid 314 to be analyzed can penetrate through the meshes, on account of their size.

The bio-molecules are detected as a result of the molecules binding to the capture molecules 311, which capture molecules 311 are immobilized at the surface of the electrodes 306. To enable them to be bound to these capture molecules, the bio-molecules to be detected have to come into active contact with the capture molecules 311. As shown in FIG. 3C, for this purpose the bio-molecules first have to penetrate through the permeation layer 303, which can be achieved, for example, by forming the permeation layer 303 as a grid.

Furthermore, for the functionality of the biochip arrangement 320, it is necessary for an electric voltage 313 to be applied between the permeation layer 303 and the reference electrode 312. If the electric voltage 313 is selected in such a way that the permeation layer is electrically positively charged on account of the electric voltage, electrically negatively charged bio-molecules which are to be detected and are contained in the liquid 314 to be analyzed are attracted by an electrical force of the permeation layer 303 and therefore move toward the permeation layer 303 (electrophoresis). By way of example, DNA strands under physiological pH values (approximately pH 6 to pH 9) in solution are electrically negatively charged. A permeation layer 303 which is under a positive electric voltage is therefore suitable for accumulating negatively charged DNA strands in the vicinity of the permeation layer 303. The increase in concentration of the molecules to be detected in the vicinity of the permeation layer 303 or in the vicinity of the capture molecules 311 improves the detection sensitivity of the biochip arrangement 320 for bio-molecules which are to be analyzed.

If, contrary to the scenario described above, an electric voltage 313 is applied between the reference electrode 312 and the permeation layer 303, so that the permeation layer 303 is electrically negatively charged, electrically positively charged bio-molecules will accumulate in the vicinity of the permeation layer 303 as a result of electrophoresis. By way of example, proteins are often electrically positively charged at physiological pH values.

The actual detection of the bio-molecules which may be present in a liquid 314 to be analyzed is effected by the bio-molecules binding to the capture molecules 311 which are immobilized at the surface of an electrode 306. The electrode 306 may be made from any desired electrically conductive material which is suitable for immobilizing the capture molecules 311, which are tailored to a specific bio-molecule to be detected, at its surface. Gold-thiol bonding has proven to be a very suitable form of bonding. Alternatively, by way of example, trichlorosilanes (SiCl₃) at end sections of capture molecules 311 can, for example, bond to silicon, aluminum or titanium surfaces. The electrically conductive material of the electrodes 306 is to be selected in such a way that the capture molecules 311 which are suitable for a specific application are immobilized thereon.

The capture molecules may be nucleic acids (RNA or DNA), peptides, proteins or low-molecular compounds. In biochemistry, the term low-molecular compounds refers to chemical compounds whose molecular size is less than or approximately 1700 Dalton (=molecular weight in grams per mole).

The capture molecules 311 are to be selected in a manner which is individually tailored to a specific bio-molecule to be detected. This is because hybridization (as diagrammatically indicated in FIG. 1B) only takes place if the capture molecules 311 and the bio-molecules to be detected which may be present in the liquid 314 to be analyzed match one another in accordance with the key-lock principle. If this is the case, the DNA strands bind to the capture molecules 311 and characteristically change a biochemical or physical parameter in such a manner that an electrical signal which has different values when hybridization has taken place than when hybridization has not taken place is tapped off between the first electrical contact-making means 307 and the second electrical contact-making means 308 with the aid of the means for recording an electrical signal 309. By way of example, the capacitance between the electrodes 306, which can be regarded as a capacitor, can be used as an electrical variable which is suitable for the abovementioned purpose. However, it should be emphasized that the binding of molecules which are to be analyzed to the capture molecules 311 does not necessarily have to be detected by an electrical signal, but rather it is also possible for optical or other parameters to be used for this purpose. By way of example, the optical transmission of the capture molecules 311 may change if bio-molecules have been bound to them.

A further alternative of the biochip arrangement according to the invention consists in using a plurality of electrically conductive permeation layers 303 which are each arranged at a predetermined distance from one another, it being possible for an electric voltage 313 to be applied to each of the permeation layers 303. In other words, a multistage increase in concentration of the bio-molecules to be detected can be achieved as a result of a plurality of permeation layers 303, for example with gradually increasing voltages 313, being connected in series. In this way, it is possible to form a concentration gradient of the bio-molecules, the concentration at a location being higher the lower the distance between this location and the active sensor surface.

The way in which the biochip arrangement according to the invention functions is to be explained in more detail below with reference to a further preferred exemplary embodiment, which is illustrated in cross section in FIG. 4A and in plan view in FIG. 4B.

FIG. 4A shows a biochip arrangement 400 having a substrate 401, seven sensors 402, a permeation grid 403, a spacer 404, a delimiting device 405, an introduction device 406, a first electrical contact-making means 407 and a second electrical contact-making means 408, electrically conductive connection means 409 and a reference electrode 410. Some of the features of the biochip arrangement 400 shown in FIG. 4A are not shown in FIG. 4B.

A liquid to be analyzed can be introduced into the cavity formed by the permeation grid 403 and the delimiting device 405 by means of the introduction device 406. An electric voltage can be applied between the permeation grid 403 and the reference electrode 410, and as a result electrophoresis causes the bio-molecules to be detected which may be present in the liquid to be analyzed to accumulate in an area surrounding the permeation grid 403. The spacer 404 sets a distance, which can be predetermined by the thickness of the spacer 404, between the permeation grid 403 and the active sensor level, i.e. the surface of the substrate 401 on or in which the sensors 402 are arranged. This distance is preferably 1 to 2 μm.

For the biochip arrangement 400 shown in FIG. 4A and FIG. 4B to be operated as a DNA sensor, each of the sensors 402 is to be selected as an electrically conductive electrode with suitable capture molecules immobilized thereon (these capture molecules are not shown in FIG. 4A, FIG. 4B). The liquid to be analyzed, including the biological macromolecules, penetrates through the meshes 411 of the permeation grid 403 (cf. FIG. 4B). In this way, the bio-molecules to be detected, for example DNA strands, come into active contact with the capture molecules on the surface of the electrodes. If the bio-molecules to be detected and the capture molecules immobilized on the surface of the electrodes are complementary to one another, hybridization takes place. This causes the electrical parameter of the capacitance between the sensors 402 provided as electrodes to change. The capacitance or any other desired parameter can be recorded between the first electrical contact-making means 407 and the second electrical contact-making means 408.

The electrical coupling between the first electrical contact-making means 407 or the second electrical contact-making means 408 and the sensors 402 is realized by electrical connection means 409. These connection means, like the sensors 402 or the electrical contact-making means 407, 408, may be integrated on the substrate 401 (for example a silicon wafer) or may be provided as separate electrical components.

As shown in FIG. 4A, the spacer 404 is secured direct to the surface of the substrate 401. The spacer 404 may, for example, be adhesively bonded securely to the surface of the substrate 401, or alternatively the spacer 404 may also be deposited on the surface of the substrate 401 by means of a semiconductor technology process. In accordance with the exemplary embodiment of the biochip arrangement 400 shown in FIG. 4A, FIG. 4B, the spacer 404 is in the form of a polyimide ring. The thickness of this polyimide ring is preferably 1 to 2μm.

The permeation grid 403 is secured, for example by adhesive bonding, to the top side of the spacer 404. As shown in particular in FIG. 4B, according to the preferred exemplary embodiment the permeation grid is configured as a grid of parallel wires which are arranged in two mutually perpendicular directions, so that meshes 411 are formed.

In accordance with the exemplary embodiment of the biochip arrangement 400 according to the invention shown in FIG. 4A, FIG. 4B, the delimiting device 405 is formed as a Plexiglass tube which is, for example, adhesively bonded to the surface of the permeation grid 403. As shown in FIG. 4B, both the spacer 404 and the delimiting device 405 are substantially in the form of hollow cylinders in accordance with the described exemplary embodiment of the biochip arrangement 400.

Finally, it should be noted that a liquid which is to be analyzed can be introduced into the biochip arrangement 400, usually in the milliliter to microliter range, using the introduction device 406. The introduction device 406 may, for example, be a pipette or a cannula.

1. A biochip arrangement for the detection of bio-molecules, comprising:
a substrate;
at least one sensor arranged on or in the substrate; and
an electrically conductive permeation layer which is arranged at a predetermined distance other than zero from the surface of the substrate and to which an electric voltage can be applied, in such a manner that bio-molecules which are to be detected can be accumulated in a region in the vicinity of the permeation layer by means of electrophoresis.

2. The biochip arrangement as claimed in claim 1, further comprising a spacer which is arranged between the substrate and the permeation layer wherein the spacer thickness is equal to the predetermined distance between the permeation layer and the surface of the substrate.

3. The biochip arrangement as claimed in claim 1 or 2, further comprising a delimiting device, the delimiting device being arranged along a continuous path on the permeation layer, in such a manner that the delimiting device and the permeation layer form a cavity.

4. The biochip arrangement as claimed in claim 1, further comprising the at least one sensor having at least one electrode, wherein each of the electrodes may be coupled to electrical contact-making means.

5. The biochip arrangement as claimed in claim 4, further comprising a multiplicity of capture molecules, which are coupled to at least one of the electrodes.

6. The biochip arrangement as claimed in claim 4 or 5, further comprising at least one electrical contact-making means, at least one of the electrodes being coupled to at least one of the electrical contact-making means, so that at least one signal can be tapped off at the electrical contact-making means.

7. The biochip arrangement as claimed in one of claims 1 or 2, further comprising a reference electrode, in such a manner that an electric voltage can be applied between the permeation layer and the reference electrode.

8. The biochip arrangement as claimed in claim 1, further comprising the permeation layer having a core which is made from an electrically conductive material.

9. The biochip arrangement as claimed in claim 8, in which the permeation layer also includes a covering which surrounds the core and is made from a porous material.

10. The biochip arrangement as claimed in claim 9, in which the porous material of the permeation layer has pores of a predetermined size, such that molecules whose size is less than or equal to the predetermined pore size can diffuse through the porous material, whereas molecules whose size exceeds the predetermined pore size cannot diffuse through the porous material.

11. The biochip arrangement as claimed in one of claims 8 to 10, in which the electrically conductive material of the permeation layer is a metal or a semiconductor.

12. The biochip arrangement as claimed in one of claims 8 to 10, in which the electrically conductive material of the permeation layer is gold.

13. The biochip arrangement as claimed in claim 8, in which the permeation layer is formed as a grid.

14. The biochip arrangement as claimed in claim 13, in which adjacent wires of the grid are arranged at a distance of approximately 100 nm from one another.

15. The biochip arrangement as claimed in one of claims 2, 3-5, 8, 9, 11, 13 or 14, in which the spacer is arranged along a continuous path on the substrate, in such a manner that the spacer and the substrate form a cavity.

16. The biochip arrangement as claimed in claim 3, in which the spacer and the delimiting device is substantially in the form of a hollow cylinder.

17. The biochip arrangement as claimed in claim 3, in which the spacer and the delimiting device is made from an electrically nonconductive material.

18. The biochip arrangement as claimed in claim 3, in which the spacer and the delimiting device is made from of the materials selected from the group consisting of glass, Plexiglass, polyimide, polycarbonate, polyethylene, polypropylene and polystyrene.

19. The biochip arrangement as claimed in claim 2 having at least one further spacer, which are arranged between the substrate and the permeation layer and the thickness of which is equal to the predetermined distance between the permeation layer and the surface of the substrate.

20. The biochip arrangement as claimed in claim 4, in which the at least one electrode is made from gold.

21. The biochip arrangement as claimed in claim 5, in which the capture molecules selected from the group consisting of are nucleic acids, peptides, proteins and low-molecular compounds.

22. The biochip arrangement as claimed in claim 1, which includes a plurality of electrically conductive permeation layers which are arranged at a predetermined distance from one another, it being possible for an electric voltage to be applied to each of the permeation layers.
